(19) United States
(12) Patent Application Publication
(10) Pub. No.: US 2022/0208378 A1
(43) Pub. Date: Jun. 30, 2022

(54) INFORMATION PROCESSING APPARATUS, INFORMATION PROCESSING METHOD, AND NON-TRANSITORY STORAGE MEDIUM

(71) Applicant: TOYOTA JIDOSHA KABUSHIKI KAISHA, Toyota-shi (JP)

(72) Inventors: Toyokazu NAKASHIMA, Nagoya-shi (JP); Makoto TAMURA, Miyoshi-shi (JP); Yusuke MITOMA, Nisshin-shi (JP); Kosuke KOTAKE, Nagoya-shi (JP); Junko KATOH, Nagoya-shi (JP); Noriko KOREYASU, Toyota-shi (JP); Hiromi TONEGAWA, Konan-shi (JP); Aiko FUJII, Nagoya-shi (JP); Yuutarou MIYASHITA, Nagoya-shi (JP)

(73) Assignee: TOYOTA JIDOSHA KABUSHIKI KAISHA, Toyota-shi (JP)

(21) Appl. No.: 17/644,694

(22) Filed: Dec. 16, 2021

(30) Foreign Application Priority Data
Dec. 24, 2020 (JP) 2020-215594

(51) Int. Cl. G16H 50/20; G16H 10/60; G16H 40/40; G06Q 30/02

(52) U.S. Cl. CPC G16H 50/20; G06Q 30/0208; G16H 40/40; G16H 10/60

(57) ABSTRACT

An information processing apparatus including a controller configured to perform receiving first information indicating that a first user who has been registered in advance performed emergency treatment of an injured or sick person, and generating data representing an incentive corresponding to the first information received.

### CROSS REFERENCE TO THE RELATED APPLICATION

This application claims the benefit of Japanese Patent Application No. 2020-215594, filed on Dec. 24, 2020, which is hereby incorporated by reference herein in its entirety.

### BACKGROUND

### Technical Field

The present disclosure relates to an information processing apparatus, an information processing method, and a non-transitory storage medium storing a program.

### Description of the Related Art

Patent Literature 1 discloses a system that is composed of a user terminal, third party terminals, and a critical care assisting device. In this system, the urgency of critical care is determined from the biological information of a user. In addition, distance information between the user and each of a plurality of third parties is also calculated. Then, a third-party terminal to which a rescue instruction information is transmitted is selected based on the urgency of critical care, the distance information, and the attribute information of each third party.

### CITATION LIST

### Patent Literature

Patent Literature 1: Japanese Patent Application Laid-Open Publication No. 2012-222443

### SUMMARY

An object of the present disclosure is to facilitate emergency activities.

One aspect of the present disclosure is directed to an information processing apparatus including a controller configured to perform:

receiving first information indicating that a first user who has been registered in advance performed emergency treatment of an injured or sick person; and

generating data representing an incentive corresponding to the first information received.

Another aspect of the present disclosure is directed to an information processing method for causing a computer to perform:

receiving first information indicating that a first user who has been registered in advance performed emergency treatment of an injured or sick person; and

generating data representing an incentive corresponding to the first information received.

A further aspect of the present disclosure is directed to a non-transitory storage medium storing a program for causing a computer to perform:

obtaining information about a first user; and

transmitting first information indicating that the first user performed emergency treatment of an injured or sick person to a server that generates data representing an incentive.

In addition, a still further aspect of the present disclosure is directed to a program for causing a computer to perform the above-described method, or a storage medium storing the program in a non-transitory manner. Moreover, a yet further aspect of the present disclosure is directed to a program stored in the non-transitory storage medium, an information processing method in which a computer executes the program, or an information processing apparatus including the non-transitory storage medium.

According to the present disclosure, emergency activities can be promoted.

### BRIEF DESCRIPTION OF THE DRAWINGS

FIG. 1 is a view illustrating a schematic configuration of a system according to a first embodiment;

FIG. 2 is a block diagram schematically illustrating an example of a configuration of each of a first terminal, a second terminal, and a server, which together constitute the system according to the first embodiment;

FIG. 3 is a diagram illustrating functional configurations of the first terminal, the second terminal, and the server;

FIG. 4 is a view illustrating by way of example a configuration of a first user information table stored in a first user information DB;

FIG. 5 is a view illustrating by way of example a configuration of an incentive information table stored in an incentive information DB;

FIG. 6 is a sequence diagram illustrating the overall processing of the system;

FIG. 7 is a flowchart of processing of providing an incentive according to the first embodiment;

FIG. 8 is a flowchart of processing in the second terminal according to the first embodiment;

FIG. 9 is a view illustrating a schematic configuration of a system according to a second embodiment;

FIG. 10 is a block diagram schematically illustrating an example of a configuration of each of a first terminal, an AED and a server, which together constitute the system according to the second embodiment;

FIG. 11 is a diagram illustrating by way of example functional configurations of the first terminal, the AED, and the server;

FIG. 12 is a flowchart of processing of providing an incentive according to the second embodiment;

FIG. 13 is a flowchart of processing in the AED according to the second embodiment;

FIG. 14 is a view illustrating by way of example a configuration of a first user information table stored in a first user information DB according to a third embodiment;

FIG. 15 is a flowchart of processing of providing an incentive according to the third embodiment;

FIG. 16 is a flowchart of processing in a first terminal according to a fourth embodiment; and

FIG. 17 is a flowchart of processing of providing an incentive according to the fourth embodiment.

### DESCRIPTION OF THE EMBODIMENTS

An information processing apparatus, which is one aspect of the present disclosure, includes a controller. The controller performs receiving first information indicating that a first user who has been registered in advance performed emergency treatment of an injured or sick person, and generating data representing an incentive corresponding to the first information thus received.

The first user is a user who holds a qualification capable of rescuing an injured or sick person, and is, for example, a doctor, a nurse, or an emergency life-saving technician (paramedic). In cases where the first user receives the incentive, the first user needs to be registered. This registration may be made by the first user. Alternatively, the registration may be made together with the obtaining of the qualification capable of rescuing an injured or sick person. The first information is generated, for example, in such a way that the first user can be identified and it can be determined that the first user has performed the emergency treatment of the injured or sick person.

The controller generates data representing the incentive corresponding to the first information. The incentive is not limited to money, but can also be related to status. For example, a credit card of the first user may be ranked up. In addition, the incentive may be an addition of points for ranking up the credit card. By providing the incentive according to the first information in this manner, it is possible to promote the first user to participate in emergency activities.

Hereinafter, embodiments of the present disclosure will be described based on the accompanying drawings. The configurations of the following embodiments are examples, and the present disclosure is not limited to the configurations of the embodiments. In addition, the following embodiments can be combined with one another as long as such combinations are possible and appropriate.

### First Embodiment

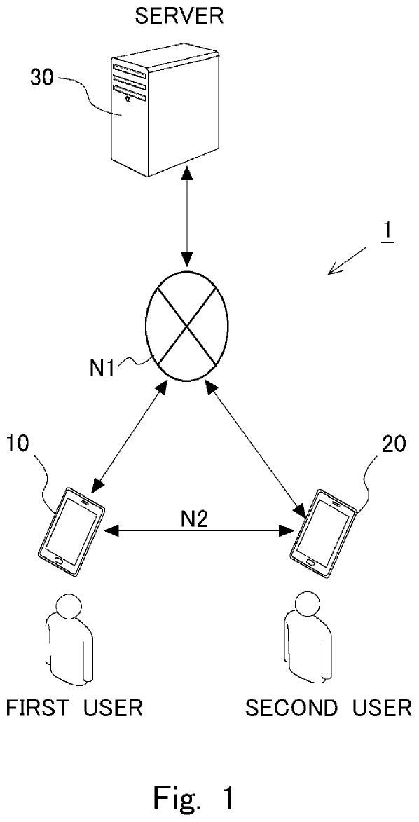

FIG. 1 is a view illustrating a schematic configuration of a system 1 according to a first embodiment. The system 1 is, for example, a system that provides an incentive to a first user when the first user performs emergency treatment on an injured or sick person. The system 1 includes a first terminal 10 used by the first user, a second terminal 20 used by a second user, and a server 30. The first user is a user who can perform emergency treatment on the injured or sick person, and is, for example, a doctor, a nurse, or a qualified medical worker such as a paramedic. On the other hand, the second user is a user who receives a request to rescue the injured or sick person and rushes to the site by, for example, an ambulance, and is, for example, a paramedic.

The first user is, for example, a user who happens to be present at the site, and who has rescued the injured or sick person at the site. Also, the first user is, for example, a user who has rescued the injured or sick person by his or her intention without receiving a request.

The first terminal 10 carried by the first user and the second terminal 20 carried by the second user can perform short-range wireless communication at the site where the injured or sick person has been rescued. Thus, information about the first user is transmitted from the second terminal 20 to the server 30. Then, the server 30 provides an incentive to the first user. The first user has been registered in the server 30 in advance. The registration of the first user may be made by the first terminal 10, for example. In addition, the first terminal 10 with which the first user makes his or her registration in the server 30 and the first terminal 10 with which the first user performs short-range wireless communication with the second terminal 20 may be different from each other.

The first terminal 10, the second terminal 20, and the server 30 are connected to one another via a network N1. Note that the network N1 is, for example, a worldwide public communication network such as the Internet or the like, and a WAN (Wide Area Network) or other communication networks may be adopted. Also, the network N1 may include a telephone communication network such as a mobile phone network or the like, and/or a wireless communication network such as Wi-Fi (registered trademark) or the like. Although one first terminal 10 and one second terminal 20 are illustrated in FIG. 1 by way of example, there can be a plurality of first terminals 10 and a plurality of second terminals 20.

In addition, the first terminal 10 and the second terminal 20 are connected to each other through a network N2. The network N2 is a short-range wireless network including, for example, Bluetooth (registered trademark), NFC (Near Field Communication), RFID (Radio Frequency Identification), BLE (Bluetooth (registered trademark) Low Energy) or the like. The first terminal 10 and the second terminal 20 can directly communicate with each other through the network N2, for example, at the site where the injured or sick person was rescued.

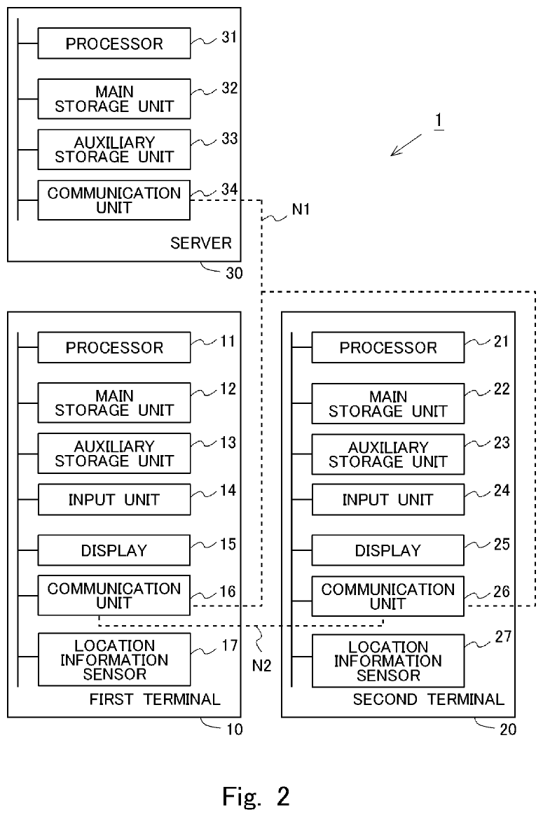

Now, hardware configurations and functional configurations of the first terminal 10, the second terminal 20, and the server 30 will be described based on FIG. 2. FIG. 2 is a block diagram schematically illustrating an example of a configuration of each of the first terminal 10, the second terminal 20, and the server 30, which together constitute the system 1 according to the present embodiment.

The server 30 has a configuration of a general computer. The server 30 includes a processor 31, a main storage unit 32, an auxiliary storage unit 33, and a communication unit 34. These components are connected to one another by means of a bus. Note that the processor 31 is an example of a controller. Also, the main storage unit 32 and the auxiliary storage unit 33 are examples of a storage unit.

The processor 31 is a CPU (Central Processing Unit), a DSP (Digital Signal Processor), or the like. The processor 31 controls the server 30 thereby to perform various information processing operations. The main storage unit 32 is a RAM (Random Access Memory), a ROM (Read Only Memory), or the like. The auxiliary storage unit 33 is an EPROM (Erasable Programmable ROM), a hard disk drive (HDD), a removable medium, or the like. The auxiliary storage unit 33 stores an operating system (OS), various programs, various tables, and the like. The processor 31 loads a program stored in the auxiliary storage unit 33 into a work area of the main storage unit 32 and executes the program, so that each component or the like is controlled through the execution of the program. As a result, the server 30 realizes functions that match predetermined purposes. The main storage unit 32 and the auxiliary storage unit 33 are computer readable recording media. Here, note that the server 30 may be a single computer or a plurality of computers that cooperate with one another. In addition, the information stored in the auxiliary storage unit 33 may be stored in the main storage unit 32. Also, the information stored in the main storage unit 32 may be stored in the auxiliary storage unit 33.

The communication unit 34 is a means or unit that communicates with the first terminal 10 and the second terminal 20 via the network N1. The communication unit 34 is, for example, a LAN (Local Area Network) interface board, a wireless communication circuit for wireless communication, or the like. The LAN interface board or the wireless communication circuit is connected to the network N1.

Here, note that a series of processing executed by the sever 30 can be executed by hardware, but can also be executed by software.

Next, the first terminal 10 and the second terminal 20 will be described. The first terminal 10 and the second terminal 20 are, for example, smartphones, mobile phones, tablet terminals, personal information terminals, wearable computers (smart watches or the like), or small computers such as personal computers (PCs). The first terminal 10 includes a processor 11, a main storage unit 12, an auxiliary storage unit 13, an input unit 14, a display 15, and a communication unit 16. These components are connected to one another by means of a bus. The second terminal 20 includes a processor 21, a main storage unit 22, an auxiliary storage unit 23, an input unit 24, a display 25, and a communication unit 26. These components are connected to one another by means of a bus. The processor 11, the main storage unit 12 and the auxiliary storage unit 13 of the first terminal 10 are the same as the processor 31, the main storage unit 32 and the auxiliary storage unit 33 of the server 30, respectively, and hence, the description thereof will be omitted. Also, the processor 21, the main storage unit 22 and the auxiliary storage unit 23 of the second terminal 20 are the same as the processor 31, the main storage unit 32 and the auxiliary storage unit 33 of the server 30, respectively, and hence, the description thereof will be omitted.

The input unit 14 is a means or unit that receives an input operation performed by the user, and is, for example, a touch panel, a mouse, a keyboard, a push button, or the like. The display 15 is a means or unit that presents information to the user, and is, for example, an LCD (Liquid Crystal Display), an EL (Electroluminescence) panel, or the like. The input unit 14 and the display 15 may be configured as a single touch panel display.

The communication unit 16 is a communication means or unit for connecting the first terminal 10 to the network N1 and the network N2. Also, the communication unit 16 is a circuit for communicating with another device (e.g., the second terminal 20, the server 30 or the like) via the network N1 or the network N2 by making use of a mobile communication service (e.g., a telephone communication network such as 5G (5th Generation), 4G (4th Generation), 3G (3rd Generation), LTE (Long Term Evolution) or the like), or a wireless communication network such as Wi-Fi (registered trademark), Bluetooth (registered trademark), NFC (Near Field Communication), RFID (Radio Frequency Identification), BLE (Bluetooth (registered trademark) Low Energy) or the like.

The input unit 24, the display 25 and the communication unit 26 of the second terminal 20 are similar to the input unit 14, the display 15 and the communication unit 16 of the first terminal 10, respectively, and hence, the description thereof will be omitted.

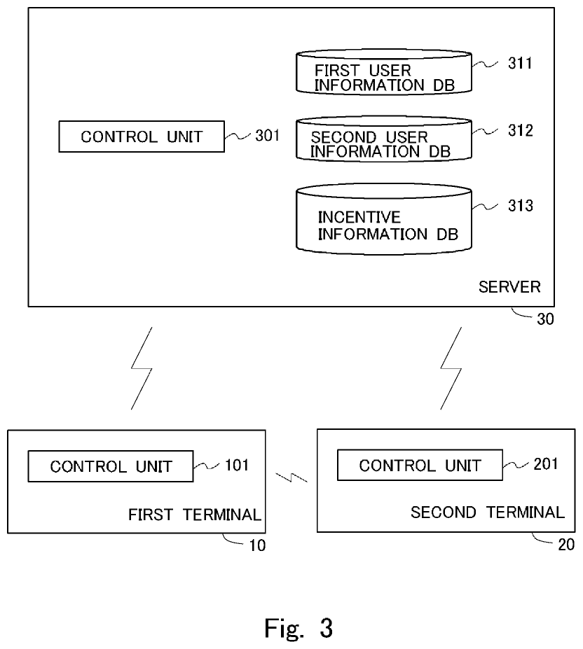

Then, FIG. 3 is a diagram illustrating functional configurations of the first terminal 10, the second terminal 20, and the server 30. The server 30 includes, as functional components, a control unit 301, a first user information DB 311, a second user information DB 312, and an incentive information DB 313. The processor 31 of the server 30 executes the processing of the control unit 301 by a computer program on the main storage unit 32. However, any of the individual functional components or a part of the processing thereof may be implemented by a hardware circuit.

The first user information DB 311, the second user information DB 312, and the incentive information DB 313 are constructed by a program of a database management system (DBMS) executed by the processor 31 to manage the data stored in the auxiliary storage unit 33. The first user information DB 311, the second user information DB 312, and the incentive information DB 313 are, for example, relational databases.

The control unit 301 receives the registration of the first user from the first terminal 10. Upon receiving the first user information from the first terminal 10, the control unit 301 stores the first user information in the first user information DB 311. The first user information includes identification information (user ID) of the first user, the registration number of the first user, and the name of the first user. The registration number is a registration number related to a medical worker.

Also, the control unit 301 receives the registration of the second user from the second terminal 20. Upon receiving the second user information from the second terminal 20, the control unit 301 stores the second user information in the second user information DB 312. The second user information includes identification information (user ID) of the second user, the registration number of the second user, and the name of the second user. The registration number is a registration number related to a medical worker.

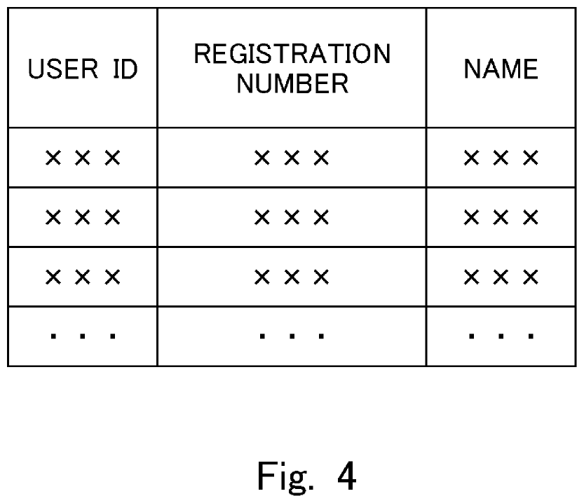

FIG. 4 is a view illustrating by way of example a configuration of a first user information table stored in the first user information DB 311. The first user information table has individual fields of user ID, registration number, and name. The user ID field is a field in which identification information (user ID) corresponding to each first user is entered. Each user ID may be assigned by the control unit 301. The registration number field is a field in which the registration number of each first user is entered. Each registration number is a number assigned to each first user when he or she registers as a medical worker. This registration number is, for example, the registration number of a doctor, which is assigned when a first user registers as a doctor. The name field is a field in which the name of each first user is entered.

A second user information table having the same configuration as that illustrated in FIG. 4 is stored in the second user information DB 312, too. Note that, for example, a paramedic can be registered as either a first user or a second user.

In addition, the control unit 301 obtains, from the second terminal 20, information about the first user who has rescued the injured or sick person at the site. The information about the first user who has rescued the injured or sick person at the site is hereinafter also referred to as rescue information. The rescue information includes the user ID of the first user. After obtaining the rescue information from the second terminal 20, the control unit 301 provides an incentive to the first user who has rescued the injured or sick person at the site. Therefore, the control unit 301 generates data representing an incentive corresponding to the information about the first user received. The incentive includes, for example, a score (or points). This score is accumulated for each first user. For example, a credit card of each first user may be ranked up according to the score. In this case, when the score thus accumulated becomes equal to or greater than a threshold value, the control unit 301 transmits information regarding the ranking up of the credit card to a credit card company. Further, for example, a discount coupon for a product may be provided in accordance with the score. Also, for example, the control unit 301 may disclose the score as information related to the status of each first user.

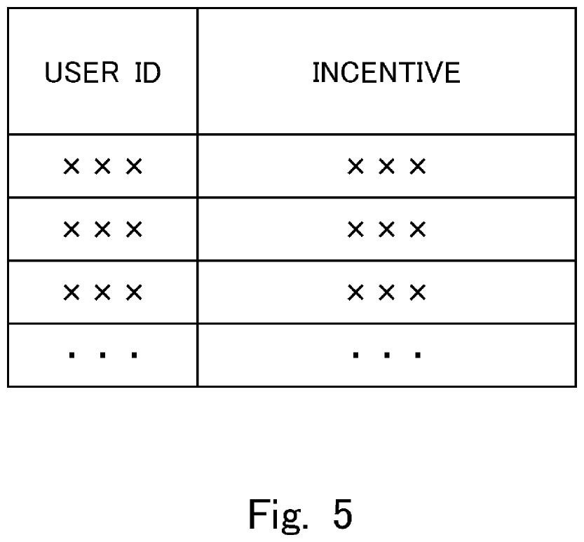

FIG. 5 is a view illustrating a configuration of an incentive information table stored in the incentive information DB 313. The incentive information table includes individual fields of user ID and incentive. The user ID field is a field in which identification information (user ID) corresponding to each first user is entered. This user ID corresponds to a user ID related to the first user information DB 311. In the incentive field, information about an incentive provided to each first user is entered. For example, upon receipt of rescue information, the control unit 301 updates the incentive field by adding a predetermined value to the score entered in the incentive field. The predetermined value may be 1, for example, or it may be set according to the content or nature of rescue in the rescue information. The content of the rescue may be obtained from the second terminal 20, or it may be obtained from information entered into a terminal of a hospital by a doctor of the hospital to which the injured or sick person is transported for emergency.

Next, the first terminal 10 includes a control unit 101 as a functional component. The processor 11 of the first terminal 10 executes the processing of the control unit 101 by a computer program on the main storage unit 12. However, a part of the control unit 101 may be executed by a hardware circuit. The control unit 101 generates first user information. The control unit 101 displays an operation screen for prompting the first user to enter or input the first user information on the display 15, and generates the first user information according to the user's input to the input unit 14. The first user information thus generated is transmitted to the server 30 by the control unit 101. For example, when user registration is performed, the first user information may be generated and transmitted to the server 30. In addition, the control unit 101 transmits the user ID to the second terminal 20 in response to a request from the second terminal 20. Also, the control unit 101 causes the display 15 to display an image corresponding to the information about the incentive provided from the server 30.

Then, the second terminal 20 includes a control unit 201 as a functional component. The processor 21 of the second terminal 20 executes the processing of the control unit 201 by a computer program on the main storage unit 22. However, a part of the control unit 201 may be executed by a hardware circuit. The control unit 201 requests the first terminal 10 of the first user who has rescued the injured or sick person to transmit the user ID. Therefore, the control unit 201 performs short-range wireless communication with the first terminal 10. Further, the control unit 201 transmits rescue information including the user ID obtained from the first terminal 10 to the server 30.

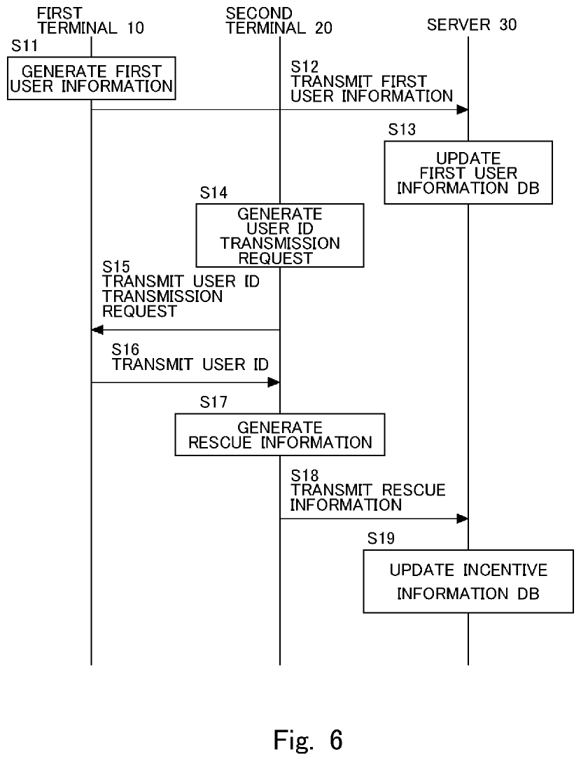

Now, the processing of the system 1 as a whole will be described. FIG. 6 is a sequence diagram illustrating the overall processing of the system 1. First user information is generated in the first terminal 10 (processing of S11), and transmitted to the server 30 (processing of S12). In the server 30 that has received the first user information, the first user information is registered in the first user information DB 311, thereby updating the first user information DB 311 (processing of S13). Note that the second user is also registered in the second user information DB 312 in the same way.

In addition, in cases where the second user recognizes, upon arrival at the site to rescue the injured or sick person, that the first user has rescued the injured or sick person at the site, the first user is requested to communicate by means of the first terminal 10 to confirm the user ID. Then, short-range wireless communication is performed between the first terminal 10 and the second terminal 20. The second terminal 20 generates information about a request to transmit the user ID (processing of S14), and transmits it to the first terminal 10 (processing of S15). In response to this request, information about the user ID of the first user is transmitted from the first terminal 10 to the second terminal 20 (processing of S16).

At the second terminal 20, which receives the information about the user ID of the first user from the first terminal 10, rescue information including the user ID of the first user and the user ID of the second user is generated (processing of S17). When the rescue information is generated, it is transmitted from the second terminal 20 to the server 30 (processing of S18). Then, in the server 30, the incentive information DB 313 is updated based on the user ID of the first user (processing of S19). That is, the server 30 updates the incentive field corresponding to the user ID of the first user by adding the score (points) at this time.

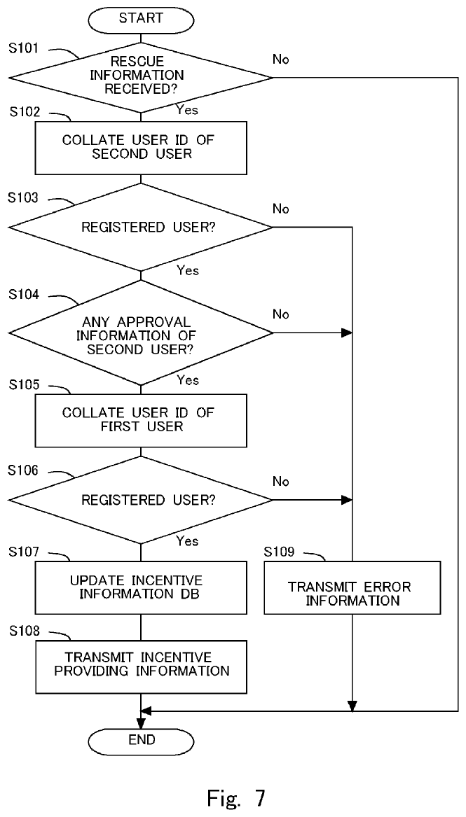

Next, processing in which the server 30 provides an incentive to the first user will be described. FIG. 7 is a flowchart of the processing of providing an incentive according to the present embodiment. The processing illustrated in FIG. 7 is repeatedly executed by the server 30 at predetermined time intervals. Here, note that the following description will be made on the assumption that necessary information has been stored in the first user information DB 311 and the second user information DB 312.

In step S101, the control unit 301 determines whether or not rescue information has been received. When an affirmative determination is made in step S101, the processing proceeds to step S102, whereas when a negative determination is made, this routine is ended. In step S102, the control unit 301 collates the user ID of the second user with the second user information DB 312. Here, in order to confirm that the second user is a registered user, the control unit 301 collates the user ID of the second user with the user IDs of second users stored in the second user information DB 312.

In step S103, the control unit 301 determines whether or not the second user is a registered user. When the user ID of the second user is present in the second user information DB 312, the control unit 301 determines that the second user is a registered user. When an affirmative determination is made in step S103, the processing proceeds to step S104, whereas when a negative determination is made, the processing proceeds to step S109.

In step S104, the control unit 301 determines whether or not the rescue information includes approval information of the second user. The approval information is generated in the second terminal 20 based on the input of the second user to the second terminal 20, and is transmitted to the server 30 together with the rescue information. The user ID of the second user may be used as the approval information. That is, when the user ID of the second user is included in the rescue information, it may be determined that the approval information of the second user is included. When an affirmative determination is made in step S104, the processing proceeds to step S105, whereas when a negative determination is made, the processing proceeds to step S109.

In step S105, the control unit 301 collates the user ID of the first user with the first user information DB 311. Here, in order to confirm that the first user is a registered user, the control unit 301 collates the user ID of the first user with the user IDs of first users stored in the first user information DB 311.

In step S106, the control unit 301 determines whether or not the first user is a registered user. When the user ID of the first user is present in the first user information DB 311, the control unit 301 determines that the first user is a registered user. When an affirmative determination is made in step S106, the processing proceeds to step S107, whereas when a negative determination is made, the processing proceeds to step S109.

In step S107, the control unit 301 updates the incentive information DB 313. The control unit 301 adds a predetermined score (or a predetermined number of points) to the score (or the number of points) in the incentive field corresponding to the first user in the incentive information DB 313, thereby to update the incentive field. In step S108, the control unit 301 transmits incentive providing information to the first terminal 10 or the second terminal 20. The incentive providing information is information about the provision of an incentive to the first user. The incentive providing information may include information about the score (or points) provided or a total score (or a total value of the points). At the first terminal 10 or the second terminal 20 that has received the incentive providing information, a screen corresponding to the incentive providing information is displayed on the display 15 or the display 25.

On the other hand, in step S109, the control unit 301 transmits error information to the second terminal 20. The error information is information about the fact that an incentive cannot be provided. The error information may include information such as the first user has not been registered, the second user has not been registered, there is no approval of the second user, or the like. At the second terminal 20 that has received the error information, a screen corresponding to the error information is displayed on the display 25.

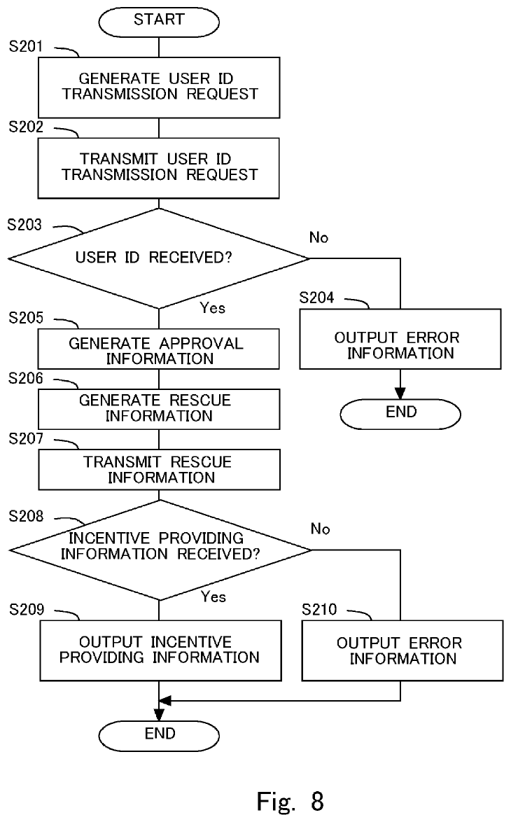

Then, processing in the user terminal 20 will be described. FIG. 8 is a flowchart of the processing in the user terminal 20 according to the present embodiment. The processing illustrated in FIG. 8 is repeatedly executed by the second terminal 20 at predetermined time intervals. Here, note that the following description will be made on the assumption that the second user ID has been registered.

In step S201, the control unit 201 generates a user ID transmission request. The user ID transmission request is information for requesting the first terminal 10 to transmit the user ID of the first user. Next, in step S202, the control unit 201 transmits the user ID transmission request thus generated to the first terminal 10.

In step S203, the control unit 201 determines whether or not the user ID of the first user has been received from the first terminal 10. When an affirmative determination is made in step S203, the processing proceeds to step S205, whereas when a negative determination is made, the processing proceeds to step S204. In step S204, the control unit 201 outputs error information to the display 25. For example, in cases where no user ID has been assigned to the first user or in cases where there is no response from the first terminal 10, an error screen is displayed.

In step S205, the control unit 201 generates approval information. The approval information may be information for approving that an incentive is provided to the first user in response to the rescue of the injured or sick person by the first user. The approval information includes, for example, the user ID of the second user or an approval code inputted or entered into the second terminal 20 by the second user. For example, the control unit 201 causes the display 25 to display a screen prompting the user to enter the user ID or the approval code of the second user, and obtains the user ID or the approval code of the second user inputted or entered into the input unit 24 by the second user. The user ID or the approval code of the second user may have been stored in advance in the auxiliary storage unit 23 of the second terminal 20.

In step S206, the control unit 201 generates rescue information. The rescue information includes the approval information, the user ID of the first user, and the user ID of the second user. In step S207, the control unit 201 transmits the rescue information to the server 30.

In step S208, the control unit 201 determines whether or not incentive providing information has been received from the server 30. When an affirmative determination is made in step S208, the processing proceeds to step S209, whereas when a negative determination is made, the processing proceeds to step S210. In step S209, the control unit 201 outputs incentive providing information. Here, for example, the control unit 201 displays a screen corresponding to the incentive providing information on the display 25. The display 25 displays, for example, that an incentive has been provided.

On the other hand, in step S210, the control unit 201 outputs error information. Here, the control unit 201 displays, for example, a screen corresponding to the error information on the display 25. For example, the fact that no incentive is provided is displayed on the display 25. In addition, it may be displayed that the first user or the second user has not been registered, or there is an error in the approval code. The information to be displayed on the display 25 is transmitted from the server 30.

Here, note that in the above description, rescue information is transmitted from the second terminal 20 to the server 30, but as an alternative, rescue information may be transmitted from the first terminal 10 to the server 30. In this case, for example, at the rescue site, the user ID and the approval information of the second user may be transmitted from the second terminal 20 to the first terminal 10, so that rescue information may be generated in the first terminal 10.

As described above, according to the present embodiment, an incentive is provided to a first user when the first user, who happens to pass by a place or location where an injured or sick person exists, rescues the injured or sick person. Therefore, it is possible to promote the first user to perform emergency activities.

### Second Embodiment

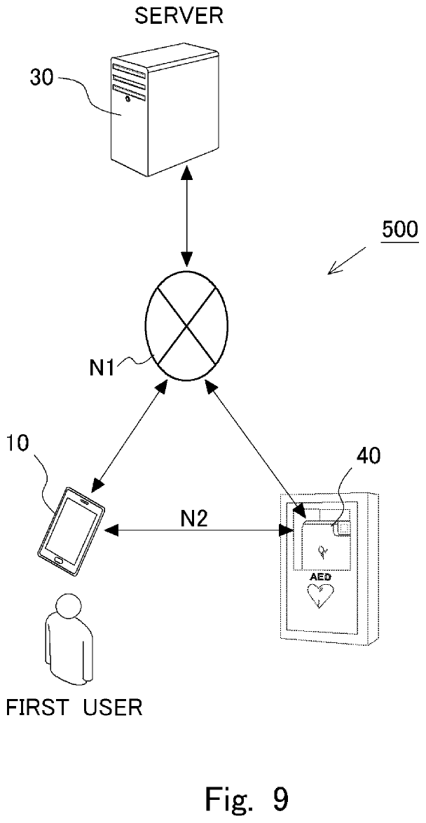

FIG. 9 is a view illustrating a schematic configuration of a system 500 according to a second embodiment. Differences of this second embodiment from the first embodiment will be mainly described. The system 500 according to this second embodiment is a system in which, when a first user rescues an injured or sick person by using an automated external defibrillator 40 (hereinafter, referred to as an AED 40), information about the first user is transmitted from the AED 40 to a server 30, so that the server 30 provides an incentive to the first user in accordance with the information.

The system 500 includes a first terminal 10 used by the first user, the AED 40 used by the first user, and the server 30. The AED 40 is installed in a public facility, for example. The first terminal 10 and the server 30 are the same as those in the first embodiment.

The first terminal 10 and the AED 40 are connected to each other through a network N2. The network N2 is a short-range wireless network including, for example, Bluetooth (registered trademark), NFC (Near Field Communication), RFID (Radio Frequency Identification), BLE (Bluetooth (registered trademark) Low Energy) or the like. The first terminal 10 and the AED 40 are able to directly communicate with each other through the network N2, for example, at a site where an injured or sick person is rescued.

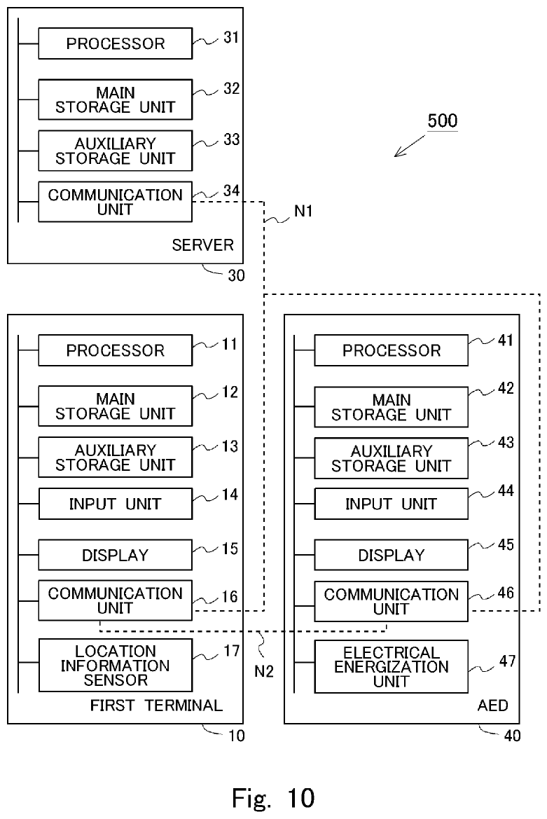

Now, hardware configurations and functional configurations of the first terminal 10, the AED 40 and the server 30 will be described based on FIG. 10. FIG. 10 is a block diagram schematically illustrating an example of a configuration of each of the first terminal 10, the AED 40 and the server 30, which together constitute the system 500 according to the second embodiment. The first terminal 10 and the server 30 are the same as those in the first embodiment, and hence, the description thereof will be omitted.

The AED 40 is a device that, when pads (electrical energization unit 47) are attached to the body of an injured or sick person, makes an electrocardiographic analysis, and performs defibrillation when ventricular fibrillation is detected. The AED 40 includes a processor 41, a main storage unit 42, an auxiliary storage unit 43, an input unit 44, a display 45, a communication unit 46, and the electrical energization unit 47. These components are connected to one another by means of a bus. The processor 41, the main storage unit 42, the auxiliary storage unit 43, the input unit 44, the display 45, and the communication unit 46 are the same as the processor 21, the main storage unit 22, the auxiliary storage unit 23, the input unit 24, the display 25, and the communication unit 26 of the second terminal 20 of the first embodiment, respectively, and hence, the description thereof will be omitted. The electrical energization unit 47 is a circuit for conducting electricity to the body of the injured or sick person, and it is provided with the pads in a part that contacts the body of the injured or sick person.

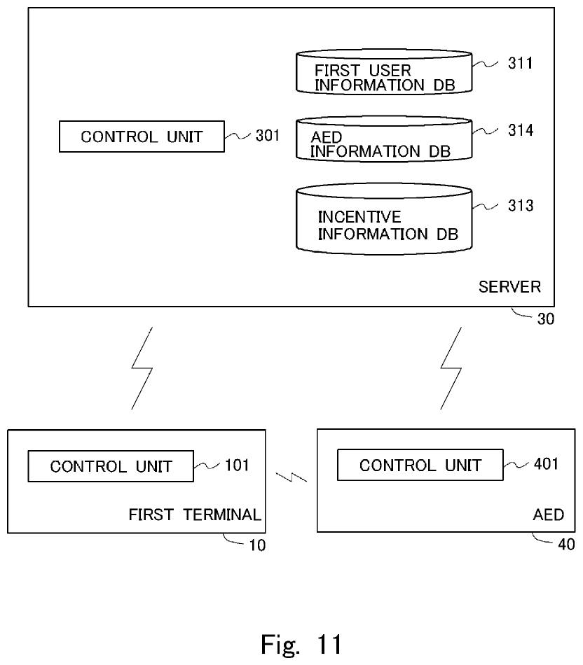

Next, FIG. 11 is a diagram illustrating functional configurations of the first terminal 10, the AED 40, and the server 30. The functional components of the first terminal 10 are the same as those of the first embodiment, and hence, the description thereof will be omitted. The server 30 includes, as functional components, a control unit 301, a first user information DB 311, an AED information DB 314, and an incentive information DB 313. That is, the AED information DB 314 is included, instead of the second user information DB 312 in the first embodiment. The AED information DB 314 stores identification information of the AED 40 (the ID of the AED 40). The AED 40 has been registered in advance by, for example, an administrator of the AED 40, and the ID of the AED 40 is given by the control unit 301 of the server 30.

The AED 40 includes a control unit 401 as a functional component. The processor 41 of the AED 40 executes the processing of the control unit 401 by a computer program on the main storage unit 42. The control unit 401 performs defibrillation based on the input of the input unit 44. Defibrillation is the same as in conventional AEDs, and the description thereof will be omitted. After defibrillation is performed, the control unit 401 requests the first terminal 10 of the first user who has rescued the injured or sick person to transmit the user ID. Therefore, the control unit 401 performs short-range wireless communication with the first terminal 10. In addition, the control unit 401 associates the user ID of the first user obtained from the first terminal 10 with the ID of the AED 40, and transmits it to the server 30 as rescue information.

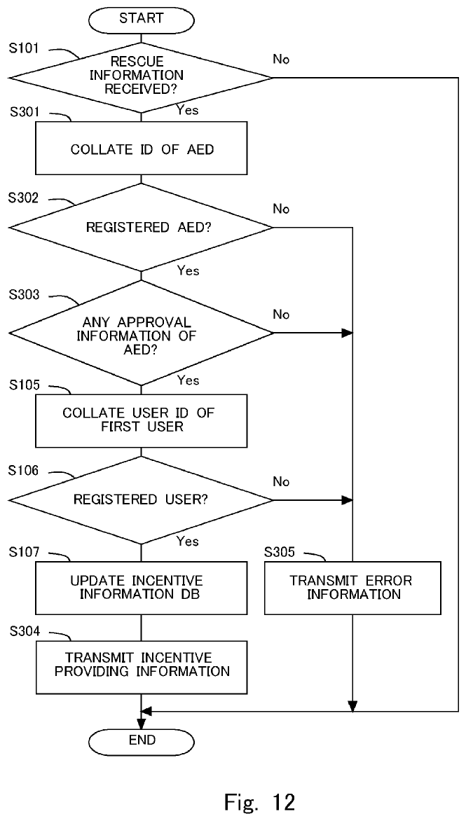

Then, processing in which the server 30 provides an incentive to the first user will be described. FIG. 12 is a flowchart of the processing of providing an incentive according to the second embodiment. The processing illustrated in FIG. 12 is repeatedly executed by the server 30 at predetermined time intervals. Here, note that the following description will be made on the assumption that necessary information has been stored in the first user information DB 311 and the AED information DB 314. Those steps in which the same processing is performed as in the routine illustrated in FIG. 7 are denoted by the same reference signs, and the description thereof will be omitted.

In the routine illustrated in FIG. 12, when an affirmative determination is made in step S101, the routine or processing proceeds to step S301. In step S301, the control unit 301 collates the ID of the AED 40 with the AED information DB 314. Here, in order to confirm that the AED 40 has been registered, the control unit 301 collates the ID of the AED 40 with the IDs of AEDs stored in the AED information DB 314.

In step S302, the control unit 301 determines whether or not the AED 40 is a registered AED. When the ID of the AED 40 is present in the AED information DB 314, the control unit 301 determines that the AED 40 has been registered. When an affirmative determination is made in step S302, the processing proceeds to step S303, whereas when a negative determination is made, the processing proceeds to step S305.

In step S303, the control unit 301 determines whether or not the rescue information includes approval information of the AED 40. When the AED 40 has been used and has communicated with the first terminal 10, the approval information is generated in the AED 40 and is transmitted to the server 30 together with the rescue information. When an affirmative determination is made in step S303, the processing proceeds to step S105, whereas when a negative determination is made, the processing proceeds to step S305.

In addition, in step S304, the control unit 301 transmits incentive providing information to the first terminal 10 or the AED 40. On the other hand, in step S305, the control unit 301 transmits error information to the AED 40. The error information is information about the fact that an incentive cannot be provided. Note that the error information may include information such as the first user has not been registered, the AED 40 has not been registered, or the like. In the AED 40 that has received the error information, a screen corresponding to the error information is displayed on the display 45.

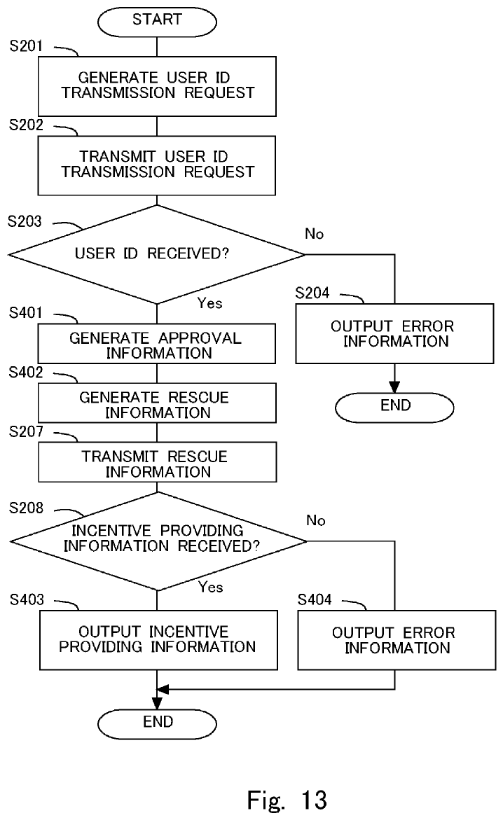

Next, processing in the AED 40 will be described. FIG. 13 is a flowchart of the processing in the AED 40 according to the second embodiment. The processing illustrated in FIG. 13 is repeatedly executed by the AED 40 at predetermined time intervals. Here, note that the following description will be made on the assumption that the ID of the AED 40 has been registered. In addition, those steps in which the control unit 401 of the AED 40 executes the same processing as that of the control unit 201 in the routine illustrated in FIG. 8 are denoted by the same reference signs, and the description thereof will be omitted.

In the routine illustrated in FIG. 13, when an affirmative determination is made in step S203, the routine or processing proceeds to step S401. In step S401, the control unit 401 generates approval information. The approval information includes, for example, the ID of the AED 40. The ID of the AED 40 has been stored in advance in the auxiliary storage unit 43. Also, the approval information includes information indicating that the user has used the AED 40.

In step S402, the control unit 401 generates rescue information. The rescue information includes the approval information and the user ID of the first user.

In step S403, the control unit 401 outputs incentive providing information. Here, for example, the control unit 401 displays a screen corresponding to the incentive providing information on the display 45. The display 45 displays, for example, that an incentive has been provided.

On the other hand, in step S404, the control unit 401 outputs error information. Here, the control unit 401 displays, for example, a screen corresponding to the error information on the display 45. For example, the fact that no incentive is provided is displayed on the display 45. In addition to this, the fact that the first user or the AED 40 has not been registered may be displayed.

As described above, according to the second embodiment, an incentive is provided to a first user when the first user, who happens to pass by a place or location where an injured or sick person exists, rescues the injured or sick person by using the AED 40. Therefore, it is possible to promote the first user to perform emergency activities.

Here, note that in the system 500 according to the second embodiment, information about the first user is transmitted from the AED 40 to the server 30, but instead of this, information about the fact that the first user has used the AED 40 may be transmitted from the first terminal 10 to the server 30 together with the information about the first user. Then, the server 30 may provide an incentive to the first user according to that information. In this case, for example, at the rescue site, the ID of the AED 40 and the approval information may be transmitted from the AED 40 to the first terminal 10, so that rescue information may be generated in the first terminal 10.

### Third Embodiment

In a system 1 according to a third embodiment, an incentive is provided to a first user when the location of the first user and the location at which an emergency activity was performed overlapped with each other in a time zone in which the emergency activity was performed. The location of the first user is obtained from the location information of the first terminal 10. The location where the emergency activity was performed may be a place or location where a rescue is requested, or may be obtained based on the location information of the second terminal 20. Alternatively, it may be obtained based on the location information of a terminal of a person in need of rescue. In the following, an example of using the location information of the second terminal 20 will be described. In the third embodiment, short-range wireless communication between the first terminal 10 and the second terminal 20 is not performed.

Here, at the site of the emergency activity, even a small amount of time cannot be wasted, so that it may be difficult to perform short-range wireless communication between the first terminal 10 and the second terminal 20. Accordingly, in cases where the location information of the first terminal 10 and the location information of the second terminal 20 overlapped with each other at the point in time at which the emergency activity was performed, the control unit 301 of the server 30 determines that the first user carrying the first terminal 10 performed the emergency activity at the site. For example, after an injured or sick person was rescued, the second user may input or enter the point in time and the location into the second terminal 20. Alternatively, for example, the server 30 may obtain a destination and an arrival point in time of an ambulance, and determine that the first user performed an emergency activity when the destination of the ambulance and the location of the first terminal 10 overlapped with each other at the arrival point in time of the ambulance. Here, note that the point in time and the location do not need to overlap exactly, but only need to be considered as being in the same place at the same time zone.

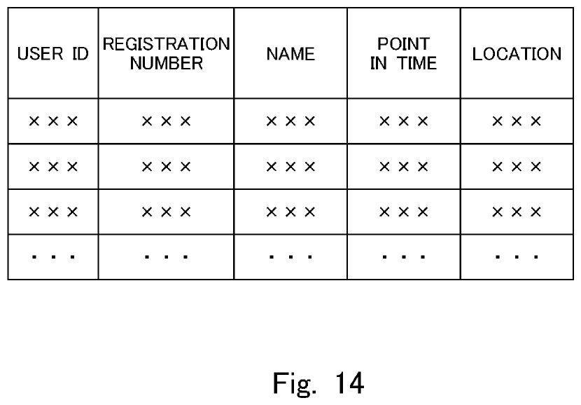

FIG. 14 is a diagram illustrating a configuration of a first user information table stored in a first user information DB 311 according to the third embodiment. The first user information table includes fields of user ID, registration number, name, point in time, and location. The fields of user ID, registration number, and name are the same as those in FIG. 4. The point in time field is a field in which a point in time is entered. The point in time may be entered at predetermined intervals. Also, instead of the point in time, a time period or zone having a certain time width may be entered. The location field is a field in which location information corresponding to a point in time is entered. In the third embodiment, first user information includes a user ID, a registration number, a name, a point in time, and a location stored in the first user information DB 311. A second user information DB 312 according to the third embodiment also stores second user information having the same configuration as that illustrated in FIG. 14.

The control unit 301 of the server 30 obtains, for example, from the second terminal 20, information about a location and a point in time at which an emergency activity was performed. For example, when the location and the point in time are entered into the second terminal 20 at the time the second user fills out a report on the emergency activity, that information is transmitted from the second terminal 20 to the server 30. Alternatively, for example, a destination of an ambulance and a point in time of arrival at the destination may be obtained from the ambulance or a server managing the ambulance as the location at which the emergency activity was performed.

Upon obtaining the location and the point in time at which the emergency activity was performed, the control unit 301 determines whether or not the first user was present at the same location at the same point in time, and if present, provides an incentive to the first user. That is, the first user who was present at the location where the emergency activity was performed is considered to have participated in the emergency activity and is given an incentive.

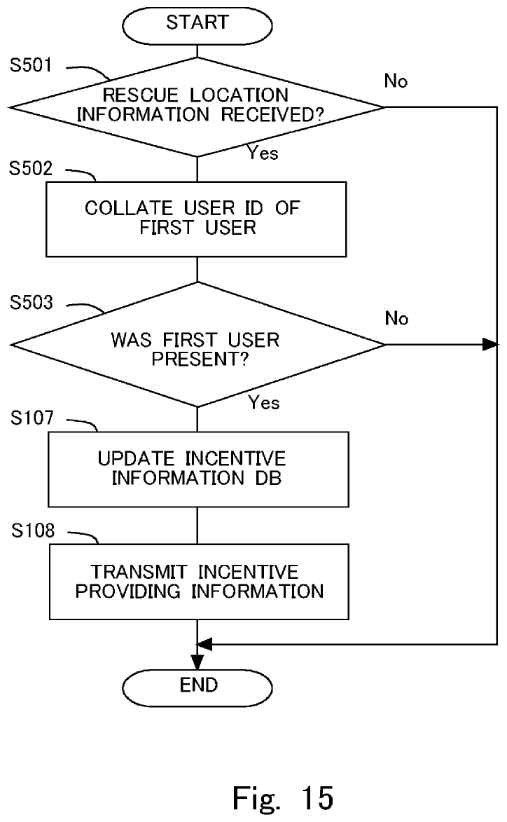

Then, processing in which the server 30 provides an incentive to the first user will be described. FIG. 15 is a flowchart of the processing of providing an incentive according to the third embodiment. The processing illustrated in FIG. 15 is repeatedly executed by the server 30 at predetermined time intervals. Here, note that the following description will be made on the assumption that necessary information has been stored in the first user information DB 311 and the second user information DB 312.

In step S501, the control unit 301 determines whether or not rescue location information has been received. The rescue location information includes information indicating the location and the point in time at which the emergency activity was performed, and is transmitted from, for example, the second terminal 20, the ambulance, the server that manages the ambulance, or the like. The rescue location includes, for example, location information indicating the location where the emergency activity was performed, such as coordinates, an address or the like. When an affirmative determination is made in step S501, the processing proceeds to step S502, whereas when a negative determination is made, this routine is ended.

In step S502, the control unit 301 identifies the first user who was present at the same location and at the same point in time as the location and the point in time at which the rescue activity was performed. The control unit 301 identifies a first user corresponding to a record in which the location and the point in time stored in the first user information DB 311 match the location and the point in time included in the rescue location information.

In step S503, the control unit 301 determines whether or not the first user was present at the location where the emergency activity was performed. The control unit 301 determines, based on the result of step S502, whether or not the first user was present at the same location and at the same point in time at which the rescue activity was performed. When an affirmative determination is made in step S503, the processing proceeds to step S107, whereas when a negative determination is made, this routine is ended.

As described above, according to the third embodiment, at a site where a first user and a second user have performed emergency activities, an incentive can be provided to the first user without causing both of the users to take time and effort.

### Fourth Embodiment

In a fourth embodiment, an injured or sick person who has been rescued approves that the first user has performed an emergency activity. Here, note that in cases where it is difficult for the injured or sick person to give approval, such as a case where the injured or sick person is unconscious, a person who was present around the injured or sick person may do so instead.

For example, short-range wireless communication may be performed between the terminal of the injured or sick person and a first terminal 10 or a second terminal 20, so that the information of the injured or sick person may be transmitted to the first terminal 10 or the second terminal 20. The information of the injured or sick person includes information for identifying the injured or sick person and information for approving that the first user has performed the emergency activity. In addition, for example, in cases where the injured or sick person has an identification card or the like to which an IC tag storing information capable of identifying the injured or sick person is attached, the information may be read from the IC tag and used as the information of the injured or sick person.

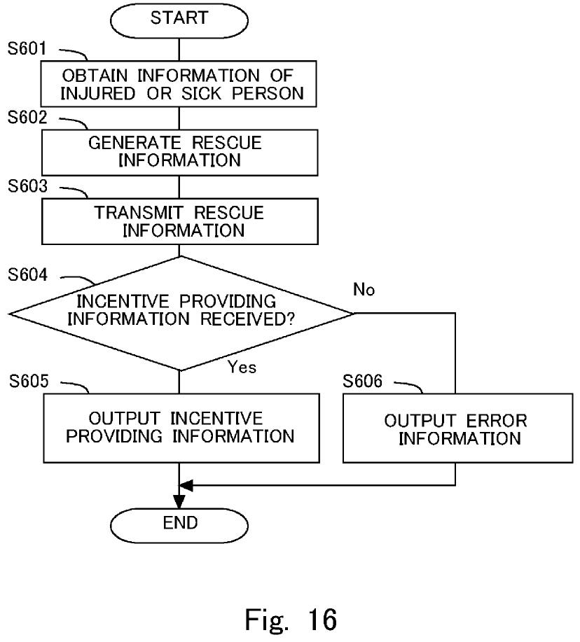

Next, processing in the first terminal 10 will be described. FIG. 16 is a flowchart of the processing in the first terminal 10 according to the fourth embodiment. The processing illustrated in FIG. 16 is repeatedly executed by the first terminal 10 at predetermined time intervals.

In step S601, the control unit 101 obtains information about an injured or sick person. For example, in cases where the injured or sick person has an IC tag storing information capable of identifying the injured or sick person, the information stored in the IC tag is read by the communication unit 16. The information thus read may be used as the information of the injured or sick person. Here, note that this information is treated as including information for approving that an incentive is provided.

In step S602, the control unit 101 generates rescue information. The rescue information includes the information for approval, the information of the injured or sick person, and the user ID of the first user. Then, in step S603, the control unit 101 transmits the rescue information to the server 30.

In step S604, the control unit 101 determines whether or not incentive providing information has been received from the server 30. When an affirmative determination is made in step S604, the processing proceeds to step S605, whereas when a negative determination is made, the processing proceeds to step S606. In step S605, the control unit 101 outputs the incentive providing information. Here, for example, the control unit 101 displays a screen corresponding to the incentive providing information on the display 15. The display 15 displays, for example, that an incentive has been provided.

On the other hand, in step S606, the control unit 101 outputs error information. Here, the control unit 101 displays, for example, a screen corresponding to the error information on the display 15. For example, the fact that no incentive is provided is displayed on the display 15.

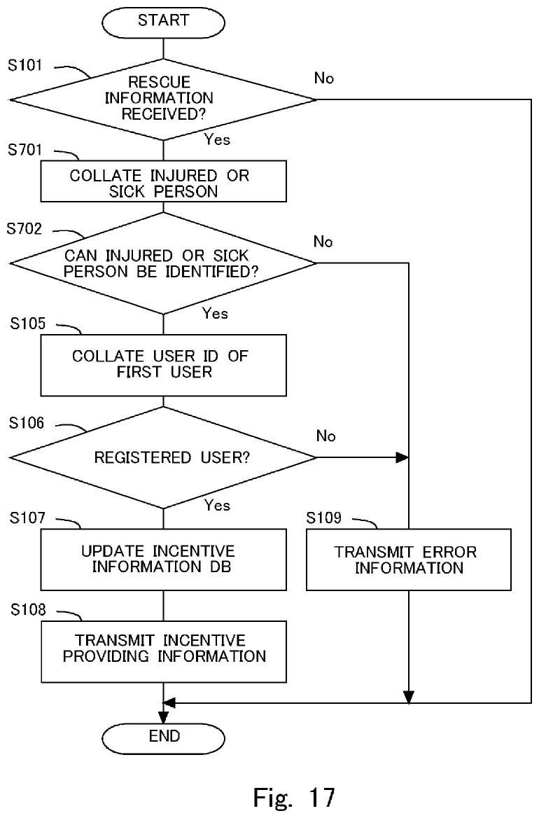

Then, processing in which the server 30 provides an incentive to the first user will be described. FIG. 17 is a flowchart of the processing of providing an incentive according to the fourth embodiment. The processing illustrated in FIG. 17 is repeatedly executed by the server 30 at predetermined time intervals. Here, note that the following description will be made on the assumption that necessary information has been stored in the first user information DB 311. Those steps in which the same processing is performed as in the routine illustrated in FIG. 7 are denoted by the same reference signs, and the description thereof will be omitted.

In the routine illustrated in FIG. 17, when an affirmative determination is made in step S101, the routine or processing proceeds to step S701. In step S701, the control unit 301 collates the information of the injured or sick person with a database. Here, for example, the control unit 301 accesses a server in which the information stored in an IC tag has been made into a database, and collates this information with the information of the injured or sick person.

In step S702, the control unit 301 determines whether or not the injured or sick person can be identified. The control unit 301 determines that the injured or sick person can be identified, when the information of the injured or sick person exists in the above database. When an affirmative determination is made in step S702, the processing proceeds to step S105, whereas when a negative determination is made, the processing proceeds to step S109.

A signature of the injured or sick person may be received as approval information from the injured or sick person. For example, the injured or sick person may enter a signature into the first terminal 10, so that the first terminal 10 may generate rescue information including this signature.

As described above, according to the fourth embodiment, an incentive is provided to a first user based on information obtained from an injured or sick person. As a result, it is possible to promote the first user to rescue the injured or sick person.

### Other Embodiments

The above-described embodiments are merely examples, but the present disclosure can be implemented with appropriate modifications without departing from the spirit thereof.

The processing and/or means (devices, units, etc.) described in the present disclosure can be freely combined and implemented as long as no technical contradiction occurs.

The processing described as being performed by one device or unit may be shared and performed by a plurality of devices or units. Alternatively, the processing described as being performed by different devices or units may be performed by one device or unit. In a computer system, a hardware configuration (server configuration) for realizing each function thereof can be changed in a flexible manner.

The present disclosure can also be realized by supplying to a computer a computer program in which the functions described in the above-described embodiments are implemented, and reading out and executing the program by means of one or more processors included in the computer. Such a computer program may be provided to the computer by a non-transitory computer readable storage medium that can be connected to a system bus of the computer, or may be provided to the computer via a network. The non-transitory computer readable storage medium includes, for example, any type of disk such as a magnetic disk (e.g., a floppy (registered trademark) disk, a hard disk drive (HDD), etc.), an optical disk (e.g., a CD-ROM, a DVD disk, a Blu-ray disk, etc.) or the like, a read-only memory (ROM), a random-access memory (RAM), an EPROM, an EEPROM, a magnetic card, a flash memory, an optical card, or any type of medium suitable for storing electronic commands or instructions.

What is claimed is:

1. An information processing apparatus including a controller configured to perform:
receiving first information indicating that a first user who has been registered in advance performed emergency treatment of an injured or sick person; and
generating data representing an incentive corresponding to the first information received.

2. The information processing apparatus according to claim 1, wherein
the first user is a user who has not received a request to perform the emergency treatment.

3. The information processing apparatus according to claim 1, wherein
the first information includes information about the fact that a paramedic, who arrived at a location where the emergency treatment was performed, approved that the first user performed the emergency treatment.

4. The information processing apparatus according to claim 1, wherein
the first information includes information about the fact that the injured or sick person on whom the emergency treatment was performed by the first user approved that the first user performed the emergency treatment.

5. The information processing apparatus according to claim 1, wherein
the first information includes information about use of an automatic external defibrillator by the first user.

6. The information processing apparatus according to claim 1, wherein
the first information includes information about a point in time and a location at which the emergency treatment was performed on the injured or sick person, and information about a location of a terminal of the first user associated with the point in time; and
the controller generates data representing an incentive corresponding to the first information, in cases where the terminal of the first user was present at a location where the emergency treatment was performed on the injured or sick person at the point in time at which the emergency treatment was performed on the injured or sick person.

7. The information processing apparatus according to claim 1, further comprising:
a storage unit configured to store information about the first user who has been registered.

8. An information processing method for causing a computer to perform:
receiving first information indicating that a first user who has been registered in advance performed emergency treatment of an injured or sick person; and
generating data representing an incentive corresponding to the first information received.

9. The information processing method according to claim 8, wherein
the first user is a user who has not received a request to perform the emergency treatment.

10. The information processing method according to claim 8, wherein
the first information includes information about the fact that a paramedic, who arrived at a location where the emergency treatment was performed, approved that the first user performed the emergency treatment.

11. The information processing method according to claim 8, wherein
the first information includes information about the fact that the injured or sick person on whom the emergency treatment was performed by the first user approved that the first user performed the emergency treatment.

12. The information processing method according to claim 8, wherein
the first information includes information about use of an automatic external defibrillator by the first user.

13. The information processing method according to claim 8, wherein
the first information includes information about a point in time and a location at which the emergency treatment was performed on the injured or sick person, and information about a location of a terminal of the first user associated with the point in time; and
the computer generates data representing an incentive corresponding to the first information, in cases where the terminal of the first user was present at a location where the emergency treatment was performed on the injured or sick person at the point in time at which the emergency treatment was performed on the injured or sick person.

14. The information processing method according to claim 8, wherein
the computer accesses a storage unit that stores information about the first user who has been registered, and collates the first user with the information.

15. A non-transitory storage medium storing a program for causing a computer to perform:
obtaining information about a first user; and
transmitting first information indicating that the first user performed emergency treatment of an injured or sick person to a server that generates data representing an incentive.

16. The non-transitory storage medium storing a program according to claim 15, wherein
the first user is a user who has not received a request to perform the emergency treatment.

17. The non-transitory storage medium storing a program according to claim 15, wherein
the first information includes information about the fact that a paramedic, who arrived at a location where the emergency treatment was performed, approved that the first user performed the emergency treatment.

18. The non-transitory storage medium storing a program according to claim 15, wherein
the first information includes information about the fact that the injured or sick person on whom the emergency treatment was performed by the first user approved that the first user performed the emergency treatment.

19. The non-transitory storage medium storing a program according to claim 15, wherein
the first information includes information about use of an automatic external defibrillator by the first user.

20. The non-transitory storage medium storing a program according to claim 15, wherein
the first information includes information about a point in time and a location at which the emergency treatment was performed on the injured or sick person, and information about a location of a terminal of the first user associated with the point in time.
